(19) United States
(12) Patent Application Publication
(10) Pub. No.: US 2026/0060559 A1
(43) Pub. Date: Mar. 5, 2026

(54) APPARATUS AND METHOD FOR MEASURING BLOOD PRESSURE VIA A FINGER

(71) Applicant: Arc Devices Limited, Dublin (IE)

(72) Inventor: Mark Haig KHACHATURIAN, Palm Beach Gardens, FL (US)

(21) Appl. No.: 19/380,887

(22) Filed: Nov. 5, 2025

Related U.S. Application Data
(63) Continuation of application No. 18/259,551, now Pat. No. 12,495,977.
(60) Provisional application No. 63/365,818, filed on Jun. 3, 2022.

(51) Int. Cl. A61B 5/022; A61B 5/00; A61B 5/0205

(52) U.S. Cl. CPC A61B 5/02241; A61B 5/02225; A61B 5/6826; A61B 5/02055

(57) ABSTRACT

There is disclosed an apparatus for facilitating measurement a person's blood pressure via the person's finger by means of an inflatable measurement bladder adapted to contact an underside of a finger defining a finger pad. A structure or other implement applies a downward force on a top portion of the finger opposite the finger pad. Blood pressure may be determined without the use of any electromagnetic radiation, such as miniature dynamic light sensing (mDLS).

### CROSS REFERENCE TO RELATED APPLICATIONS

This application is a continuation of U.S. patent application Ser. No. 18/259,551, filed Jun. 27, 2023, entitled “Apparatus and Methods for Measuring Blood Pressure and Other Vital Signs Via a Finger” which is a U.S. § 371 national stage application of PCT Application No. PCT/US2023/024253, filed Jun. 2, 2023, entitled “Apparatus and Methods for Measuring Blood Pressure and Other Vital Signs Via a Finger”, which claims the benefit of U.S. Provisional Patent Application No. 63/365,818, filed Jun. 3, 2022, entitled “Apparatus and Methods for Improving Blood Pressure Measurements Via a Finger” (“the Applications”). The Applications are incorporated herein by reference in their entireties as if the contents thereof had been stated herein.

Subject to the following clarifications and qualifications, the following U.S. Patents and U.S. Patent Publications (collectively “the References”) are also incorporated herein by reference in their entireties as if the contents thereof had been stated herein. No subject matter of the References that is contrary to the instant disclosure is incorporated herein. No claims of the References are incorporated herein. In the event of inconsistencies between this disclosure and the References, the References should be considered supplementary hereto, and the instant disclosure controls in the event of any irreconcilable inconsistencies. Information in the References is incorporated herein only to the extent that no conflict exists between such information and this disclosure. In the event of a conflict that would render any claim hereof invalid, then such conflicting information is specifically not incorporated by reference herein. The foregoing disclaimers do not apply to the Applications. The References are: U.S. Pat. Nos. 8,950,935; 10,492,684; 10,485,431; and 11,504,014; and, U.S. Published Patent Application No. 2018-0235478A1.

### TECHNICAL FIELD

This disclosure relates generally to a system for measuring blood pressure alone or in combination with one or more other non-blood pressure vital signs such as hematocrit, total protein, blood glucose levels, SpO₂, pulse rate, respiratory rate, temperature, EEG, and others, in a mammal, such as a human, via a finger. More particularly, this disclosure relates to an improved apparatus for accurately measuring blood pressure alone via the finger, or in combination with other vital signs.

### BACKGROUND

As used herein, the terms monitor/monitoring, measure/measuring, capture/capturing, detect/detecting, and sense/sensing are used synonymously, unless the context in which they are used suggests otherwise. Likewise, the terms user, person and subject, pulse rate and heart rate, pulse oximetry and SpO₂, pump and pneumatic engine, and physiological characteristics and vital signs are used synonymously, unless the context in which they are used suggests otherwise. Accordingly, and subject to the foregoing exception, such terms may be considered to have been used interchangeably throughout.

The above referenced, commonly assigned, U.S. Pat. No. 11,504,014 (“the 014 patent”) discloses an apparatus and method for measuring blood pressure and other vital signs via a finger. The blood pressure measurement system disclosed in the 014 patent employs a circular inflatable measurement bladder that is adapted to receive, and when inflated, substantially fully envelope and substantially fully compress and contact substantially the entirety of the periphery of the portion of the finger, and negates any need for the use of light, light sensors, optical measurements, or the use of any other type of electromagnetic radiation (or measurement thereof) to provide an indication of blood pressure. However, it has been found that the accuracy of blood pressure measurements taken via a finger may be improved without the use of an inflatable measurement bladder that fully surrounds or fully contacts and fully compresses the entirety of the periphery of the finger. In particular, it has been found that blood pressure measurements are unexpectedly and dramatically improved when the inflatable measurement bladder is configured to contact only a bottom portion of the finger, and a force or pressure is applied to the top of finger so as to urge the finger toward the inflatable measurement bladder while a blood pressure measurement is being taken.

### BRIEF DESCRIPTION OF THE DISCLOSURE

There is disclosed a vital sign measuring device (VSMD) that measures blood pressure via a finger. The VSMD comprises an arcuate inflatable measurement bladder adapted to only partially contact a surface of the finger, and particularly at least the surface of the finger pad on the underside of the finger but not the entirety of the surface of the finger. The bladder may be disposed on an arcuate cradle coupled to a base that houses an inflation pump, a relief valve, a pressure sensor, a microprocessor, and circuitry for controlling the pump and valve and processing signals from the pressure sensor. During a blood pressure measurement cycle, the bladder does not fully envelope the finger, or fully compress or fully contact the entirety of the periphery of the finger. Rather, the bladder contacts only a portion of the underside of the finger.

The term “structure or other implement” (SOI), and like terms used herein, mean structure that is associated or integral with the overall measurement apparatus described herein for applying a force or pressure to the top of the finger, or other structure of implement separate and/or distinct from the overall measurement apparatus, that applies such force or pressure. As disclosed herein, SOI's are employed to urge the finger pad against a corresponding portion of a bladder. The SOI enhances contact between the digital arteries in the finger pad and a corresponding portion of the bladder and reduces or eliminates movement of the finger relative to the bladder during a measurement cycle. The 014 patent describes how an oscillometric signal provided by a pressure sensor that reads bladder pressure is analyzed to provide an indication of blood pressure. The instant disclosure employs the same oscillometric signal to calculate an indication of blood pressure. The same blood pressure measurement algorithms disclosed in the 014 patent may be employed in connection with the instant disclosure. The use of an SOI has been found to significantly increase the amplitude of the oscillometric signal measured by the pressure sensor and to reduce noise and other artifacts that might otherwise be present therein, thus resulting in significantly improved signal to noise ratio and more accurate and repeatable systolic and diastolic blood pressure measurement.

The VSMD may also measure pulse rate via the oscillometric signal. There is no need for the use of light, light sensors, optical measurements, or the use of any other type of electromagnetic radiation (or measurement thereof) to provide an indication of blood pressure or pulse rate.

There is also disclosed VSMD embodiments that, in addition to measuring blood pressure via the finger pad, comprise a physiological light monitoring system (PLMS) for additionally measuring one or more of the following non-blood pressure vital signs: blood glucose levels, heart rate variability, respiration rate, SpO₂, blood flow, total hemoglobin (SpHb), PVi, methemoglobin (SpMet), acoustic respiration rate (RRa), carboxyhemoglobin (SpCO), oxygen reserve index (ORi), oxygen content (SpOC), hematocrit (Hc), total protein (TP), EEG and temperature. Details of the PLMS may be found in the 014 patent.

A display may provide visual indications of vital signs. Data may be communicated to a smartphone equipped with an app for generating and displaying health scores and for communicating vital sign data to a remote patient monitoring system.

### BRIEF DESCRIPTION OF THE DRAWINGS

The description of the drawings below refers to various embodiments of apparatuses, systems and methods for implementing a VSMD that employs an SOI for measuring blood pressure via a finger pad, and optionally a PLMS for measuring vital signs other than blood pressure and is not intended to limit the scope of the disclosure and/or the inventions described therein, except as set forth in the appended claims.

FIG. 1 is a perspective view of a VSMD for measuring blood pressure and pulse rate via a finger pad. The embodiment of FIG. 1 employs an arcuate cradle having an inflatable bladder and a strap for implementing the SOI.

FIG. 2 is a bottom view of the VSMD of FIG. 1 and illustrates a display thereof.

FIG. 3 illustrates three different regions of the bottom of a finger, i.e., finger pads, from which blood pressure and pulse rate can be measured according to the instant disclosure.

FIG. 4A illustrates the VSMD of FIG. 1 including a block diagram of the components that may be contained in the base thereof for calculating blood pressure and pulse rate.

FIG. 4B illustrates a finger resting in the cradle of the VSMD of FIGS. 1 and 4A and shows an SOI applied across the top of the finger.

FIG. 5 illustrates the VSMD of FIG. 1 with an optional PLMS.

FIG. 6 illustrates the placement of light emitters and detectors used by the PLMS of FIG. 5.

FIG. 7 is a block diagram of various sensors, switches, and circuitry that may be employed in the PLMS.

FIGS. 8A and 8B illustrate a VSMD for measuring blood pressure that employs another embodiment for implementing an SOI.

FIGS. 9A, 9B and 9C illustrate a VSMD for measuring blood pressure that employs yet another embodiment for implementing an SOI.

FIGS. 10A and 10B illustrate a VSMD for measuring blood pressure that employs still another embodiment for implementing an SOI.

FIGS. 11A and 11B illustrate a VSMD for measuring blood pressure that employs still a further embodiment for implementing an SOI.

FIGS. 12A, 12B and 12C illustrate a VSMD for measuring blood pressure that employs still a further embodiment for implementing an SOI.

FIG. 13 illustrates a VSMD for measuring blood pressure that employs still a further embodiment for implementing an SOI.

FIG. 14 illustrates a method of processing digital pressure sensor data to obtain blood pressure indications.

FIGS. 15A and 15B illustrate an oscillometric waveform and an oscillometric waveform envelope, respectively.

FIGS. 16A and 16B are graphs exemplifying the differences in amplitude of the oscillometric pressure waveform signal with and without the use of an SOI, respectively.

FIG. 17 illustrates a method for measuring Hc and TP via the PLMS.

FIG. 18 illustrates a method for measuring glucose and other physiological characteristics via the PLMS.

### DETAILED DESCRIPTION

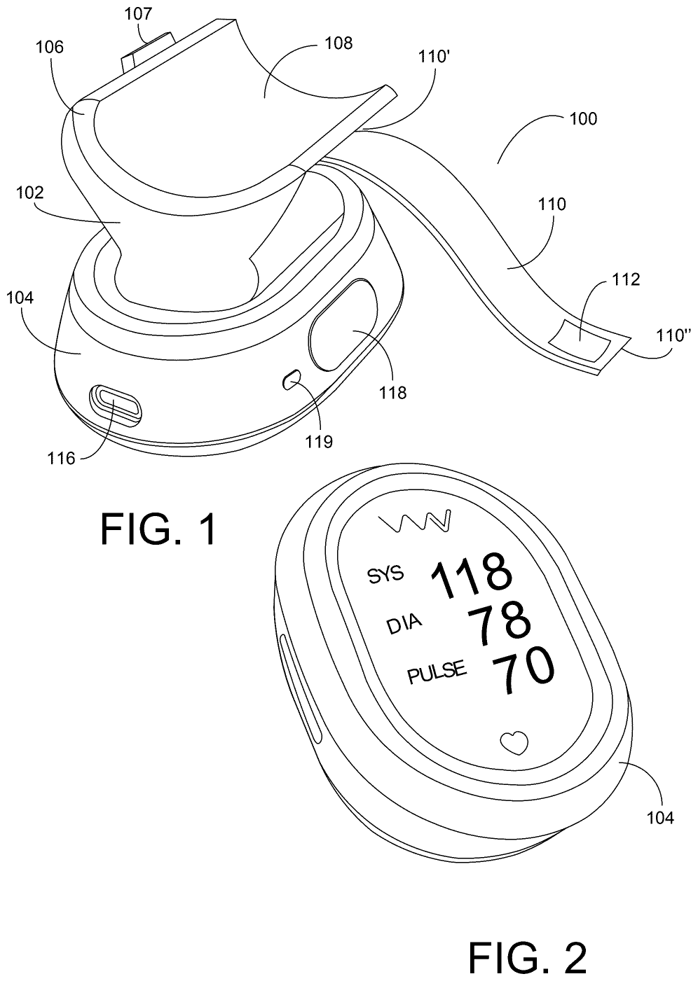

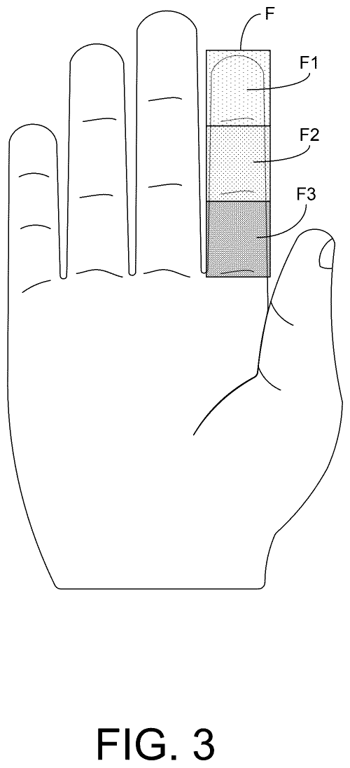

Referring to the drawings, wherein like numerals represent like elements, there is shown in FIG. 1 an embodiment of a VSMD 100 for measuring blood pressure and pulse rate. The VSMD 100 comprises a cradle 102 affixed to a base 104. The cradle 102 comprises a rigid arcuate portion 106 (which in the embodiment of FIG. 1 forms less than a full circle) to which a conforming, correspondingly arcuate inflatable bladder 108 is affixed. The cradle is adapted to receive the bottom portion of a finger defining a finger pad F1, F2 or F3 (see FIG. 3) such that the finger pad rests on the outer surface of the inflation bladder. A strap 110 defines the SOI. A proximal end 110′ of the strap is affixed to the cradle and a distal end 110″ is loose. The strap, or a portion thereof, may comprise a hook and latch fastener 112, such as Velcro®.

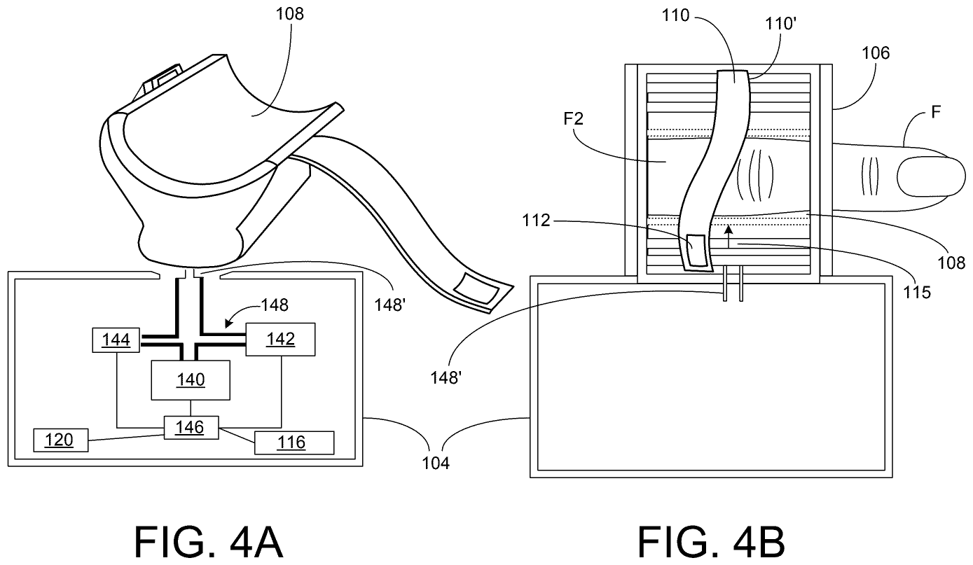

As shown in FIGS. 4A and 4B, a hose connection 148′ in the base 104 allows for pneumatic communication with the bladder 108. The base 104 also houses an inflation pump 144 for controllably inflating the bladder via the air hose 148, a pressure sensor 140 for providing indications of bladder pressure applied to the finger pad, and a relief valve 142 for controllably deflating the bladder via the air hose 148. The base also houses a temperature sensor 120 (such as an infrared sensor, thermopile, or thermocouple) for measuring body temperature at the finger, and circuitry, including a microprocessor 146. The microprocessor is operatively coupled to/interfaced with the pump, the pressure sensor, relief valve and temperature sensor (and other sensors, switches and control devices disclosed herein) for receiving pressure sensor and temperature data, controllably operating the pump and relief valve, and calculating blood pressure and body temperature measurements, and, if desired pulse rate, using the algorithms described below, and more fully in the 014 patent. A USB communications port and/or wireless communications circuitry (e.g., Bluetooth) 116 may be provided for facilitating firmware updates and/or for offloading data from the VSMD to another device, e.g., a smartphone, and/or an apparatus such as a remote patient monitoring system (RPMS). A power button 118 may be provided for powering the device up/down and/or for commencing a blood pressure measuring cycle. An LED 119 may indicate the power status (on/off) of the VSMD. A proximity sensor (not shown) may be employed to detect the presence of a finger on the bladder so as prevent inflation of the bladder unless a finger has been disposed thereon. The cradle 102 and base 104 may be comprised of molded thermoplastic materials. The bladder 108 may be constructed from an elastic air-tight material, such as silicon, nylon, latex, rubber, etc. Measurement may be performed at any of the finger pad portions F1, F2, F3 of a finger F illustrated in FIG. 3. As shown in FIG. 2, a display may be provided on the underside of the base 104 of VSMD 100 for displaying systolic blood pressure, diastolic blood pressure, pulse rate and any other desired characteristics that are being measured.

FIG. 4B illustrates a finger pad portion F2 of finger F in the cradle 106 and resting on the top (outwardly facing portion) of the bladder 108. As shown, the strap 110 has been placed over the corresponding portion of the top of the finger F and the fastening portion 112 at the distal end 110″ thereof has been affixed to a mating portion (not shown) disposed on the side of the cradle opposing the side to which the proximal end 110′ of the strap has been affixed. Alternatively, the cradle may comprise a slotted structure 107 on the side opposing the affixed strap portion 110′. In such case, the strap 110 may be placed over the finger F, through the slot in structure 107 and looped back over the finger and attached to itself in well-known fashion. The strap should be slightly tightened over the finger only insomuch as is required to prevent movement of the finger relative to the bladder and to secure the finger in the cradle. It will be appreciated that, as shown by arrow 115, inflation of the bladder 108 will apply pressure only to a portion of the underside of the finger, e.g., to the finger pad F2, since the finger has been restrained relative to the bladder by the strap 110, thereby allowing blood pressure measurement via the finger pad.

As a consequence of the combined structures of the cradle 102, the bladder 108 and strap 110, and the circuitry and algorithms described herein and in the 014 patent, there is no need to employ electromagnetic radiation (EMR) based measurement techniques, including laser based or other optical based measuring techniques (such as mDLS) that employ measurement of EMR reflection and/or transmission to measure blood pressure. Together with such structure, and the use of the oscillometric method described herein to analyze pressure data in the bladder, blood pressure may be measured with substantially greater accuracy than with the embodiments disclosed in the 014 patent, or any other known prior art systems for measuring blood pressure via a finger. Supplemental EMR based measurements that purport to improve the accuracy of blood pressure and pulse rate measurements are not needed.

As shown in FIG. 1, the cradle may be affixed to or integral with the base. However, the cradle may be separate from the base and coupled thereto via the air hose 148′ and any other electrical connection that may be required to measure other characteristics, such as temperature or those measured by the PLMS. Alternatively, a cradle may not be employed at all, and the bladder may be affixed to the finger via a strap such as strap 110 so that the bladder contacts the finger pad. The strap may tightened only so much as is needed to prevent the bladder from moving relative to the finger.

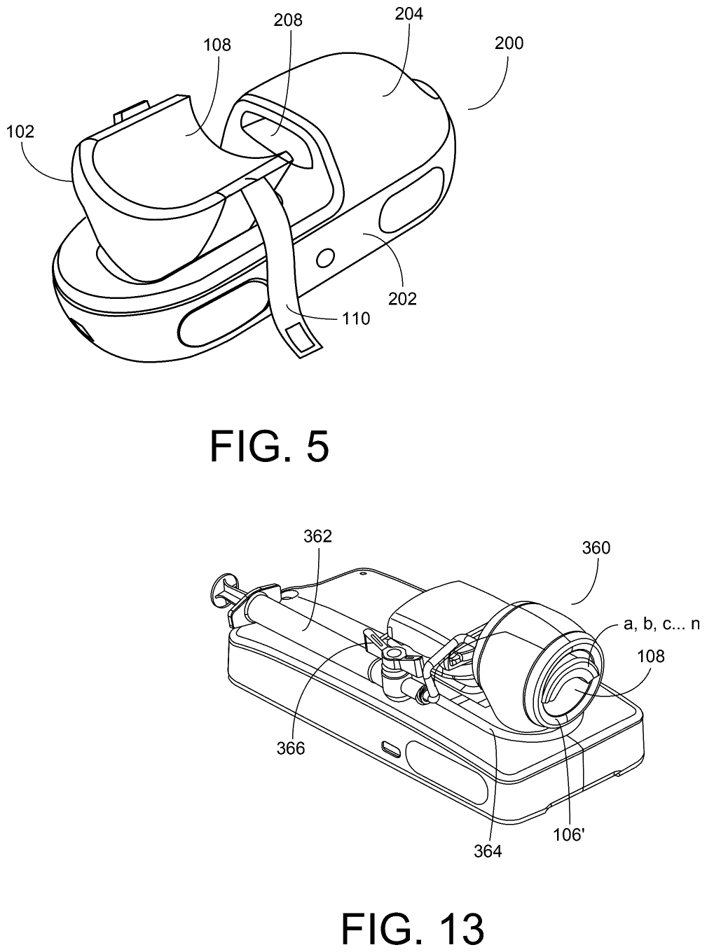

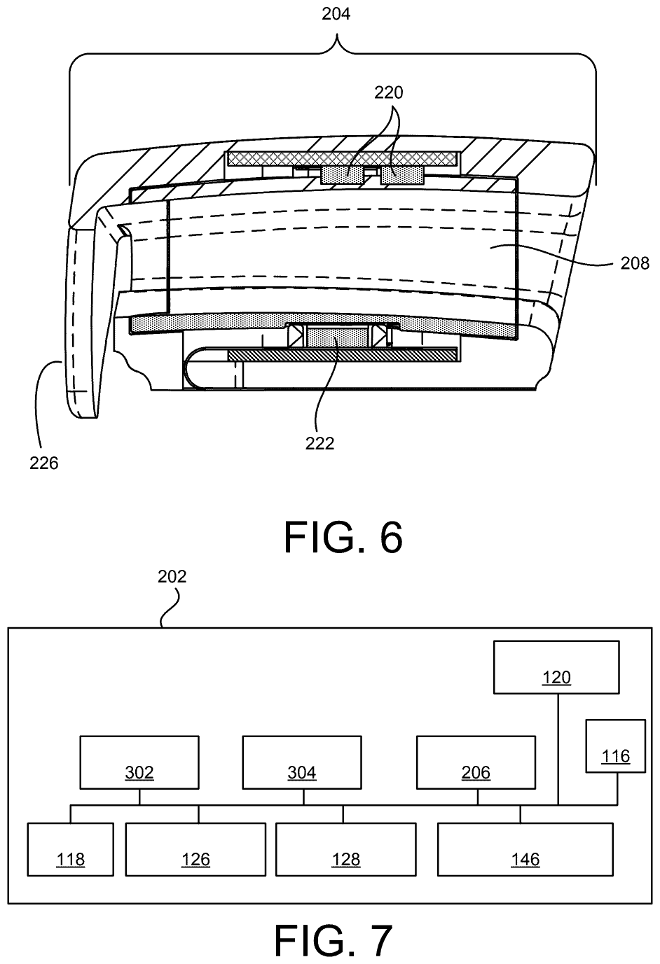

FIGS. 5, 6 and 7 illustrate a VSMD 200 that employs both the blood pressure measuring system described above, and a PLMS 204, both attached to a base 202. The PLMS may calculate vital signs and physiological characteristics such as blood glucose levels, respiration rate, saturation of peripheral oxygen (SpO₂), blood flow, total hemoglobin (SpHb), pleth variability index (PVi), methemoglobin (SpMet), acoustic respiration rate (RRa), carboxyhemoglobin (SpCO), oxygen reserve index (ORi), oxygen content (SpOC), EEG, hematocrit (Hc) and total protein (TP), as described in the 014 patent. If desired, the PLMS may also be used to calculate heart rate. The base may include a display for displaying one or more of the vital signs/physiological characteristics. Measurements are carried out after inserting one's finger F into the cavity 208 of the PLMS until the fingertip reaches a fingertip stop 226 (FIG. 6). The References provide greater details as to how such structures may be implemented and such measurements may be carried out.

As shown in FIG. 6, disposed within the PLMS are emitters 220 and emitter/detector 222 that emit/detect light as described below for obtaining data from which indications of these vital signs and physiological characteristics can be calculated. Emitters 220 and emitter/detector 222 are employed for transmissive light measurements (i.e., for measuring light emitted from emitters 220 and detected by emitter/detector 222 after being transmitted through a fingertip), whereas only emitter/detector 222 is employed for reflective light measurement (i.e., for emitting light, and measuring the light reflected from a fingertip).

Emitters 220 preferably emit light in two ranges:
a. Configuration A: 640 nm-680 nm (and preferably at about 660 nm); and 920 nm to 960 nm (and preferably at about 940 nm).
b. Configuration B: Continuous emitter from 200 nm to 1200 nm.
c. Configuration C: 12 discrete emitters at specific frequencies in the range of 200 nm to 1200 nm.

Emitter/detector 222 preferably emits light in three ranges:
a. Configuration A: 300 nm-415 nm (and preferably at about 395 nm); 640 nm 680 nm (and preferably at about 660 nm); and 920 nm-960 nm (and preferably at about 940 nm).
b. Configuration B: Continuous emitter from 200 nm to 1200 nm
c. Configuration C: 12 discrete emitters at specific frequencies in the range of 200 nm to 1200 nm.

Emitter/detector 222 preferably detects light in the range of 200-1200 nm.

Preferably, the photodiodes are physically arranged such that the emitters 220 are just above the fingernail and emitter/detectors 222 are just below the part of the bottom of the finger (the pad of the finger) under the fingernail. Further details are described in the 014 patent and the other References. The scope of the invention and the relevant appended claims are not limited to the foregoing emitter and emitter/detector configurations. Other configurations that emit and detect light within the disclosed ranges (or within reasonable proximity of the disclosed ranges) are contemplated to be within the scope of the claims, except as expressly noted herein.

As shown in FIG. 7, the base 202 of VSMD 200 may house a temperature sensor 120 for measuring body temperature at the finger, EKG pads 302, 304 for measuring EKG via one's fingertips, a camera module 126 for obtaining images of a portion of the skin of one's head when the VSM 200 is held adjacent the face, and a three-axis accelerometer 128 for detecting movement of the VSMD. The accelerometer may be used to terminate a respiratory rate measurement (or other measurement) upon detecting movement. The accelerator may also be employed for fitness tracking, e.g., measuring steps walked. A power switch 118 and a USB port 116 may also be provided. The display 206 may be integrated into the base. The 014 patent contains further details relating to the construction of and methods employed by the PLMS.

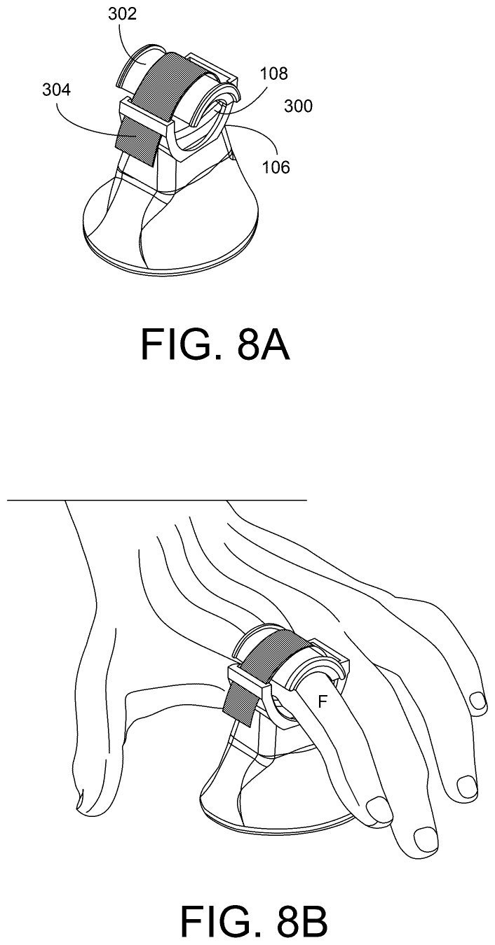

FIGS. 8-13 illustrate various alternative embodiments for implementing an SOI that may be employed in place of the SOI implementation of FIG. 1.

The VSMD 300 of FIG. 8A employs an SOI implementation that comprises an arcuate cap or insert 302 with a strap 304 disposed thereover. The cap 302 may be constructed of foam or thermoplastic. Strap 304 may be a hook and latch fastening strap. Finger F may be placed through the aperture defined by the combination of the cradle 106 and the cap 302, so that a finger pad thereof rests on the bladder surface 108, as shown in FIG. 8B. The strap 304 may be tightened and fastened in the same manner as the strap 110 in the embedment of FIG. 1. A variety of caps 302 having differing radii may be provided to accommodate varying finger sizes.

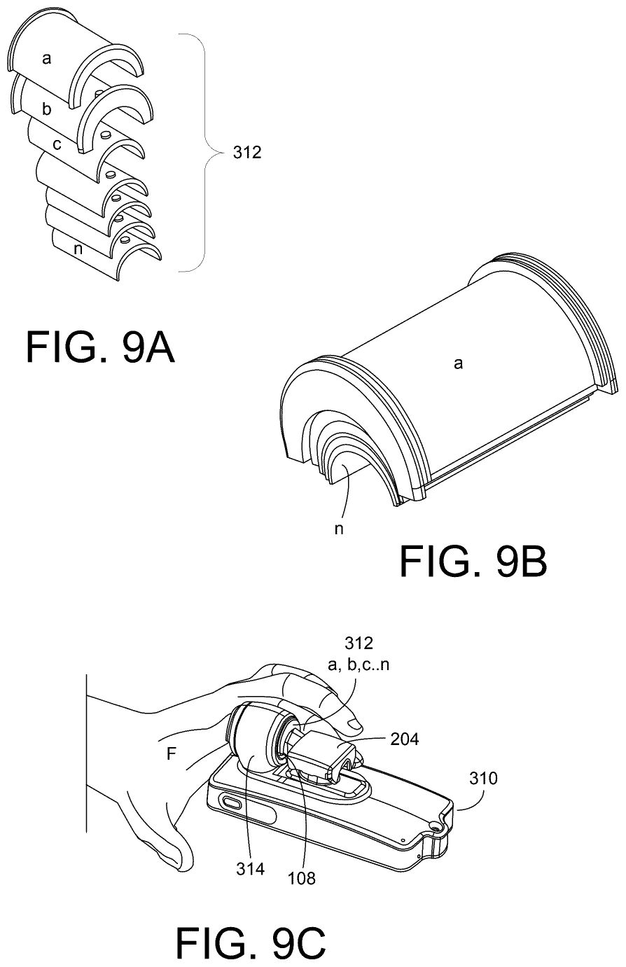

The VSMD 310 of FIGS. 9A-9C employs an SOI implementation that comprises a variety of stackable shims 312a-312n, with each shim a-n having a radius that complements the radius of each preceding and succeeding shims in the stack 312. FIG. 9C illustrates the inclusion of a PLMS 204 in the VSMD 310 for exemplary purposed only. In addition, the VSMD of FIG. 9C illustrates a substantially 360°, i.e., substantially fully circular, cradle 314 having a cavity through which the finger F may be placed. Finger F may also be extended into the PLMS 204 for vital sign measurements in addition to blood pressure. Bladder 108 is disposed at the bottom arcuate portion of the cradle, as in FIG. 1. One or more of the shims a-n may be placed on top of the finger and the finger may be then inserted into the cavity with the shims thereupon such that the shims apply the downward pressure or force previously described. Alternativity, the finger may be inserted into the cavity first, and then one of more of shims a-n may be slid on top of the finger and into the cavity so that they apply the aforementioned downward pressure or force.

Variations of both techniques may also be employed.

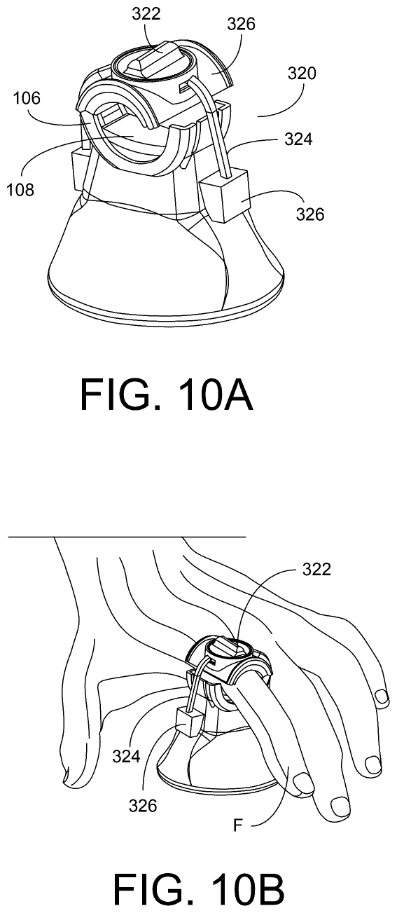

The VSMD 320 of FIGS. 10A, 10B employs an SOI implementation that comprises a cap/shim 326 to apply the downward pressure/force via a strap 324 affixed to a portion 326 of the VSMD. A twistable/rotatable knob 322 may be employed to adjust the amount of downward pressure/force to the top of Finger F.

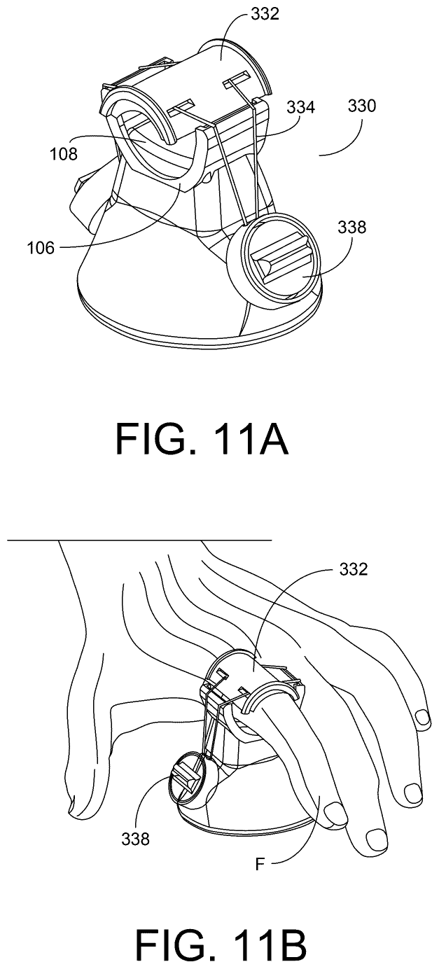

The VSMD 330 of FIGS. 11A, 11B employs an SOI implementation similar to that of FIGS. 10A, 10B. Dual straps 334 apply the downward force via a shim/cap 332 by rotation of the twistable/rotatable knob 338 disposed on the side of the base of the VSMD.

In each of the embodiments described above, it has been found that optimal results are achieved when the SOI is adjusted so as to limit lateral movement of the finger pad to a range of about 0.4 mm-4 mm relative to the surface of the inflatable bladder that contacts the finger pad.

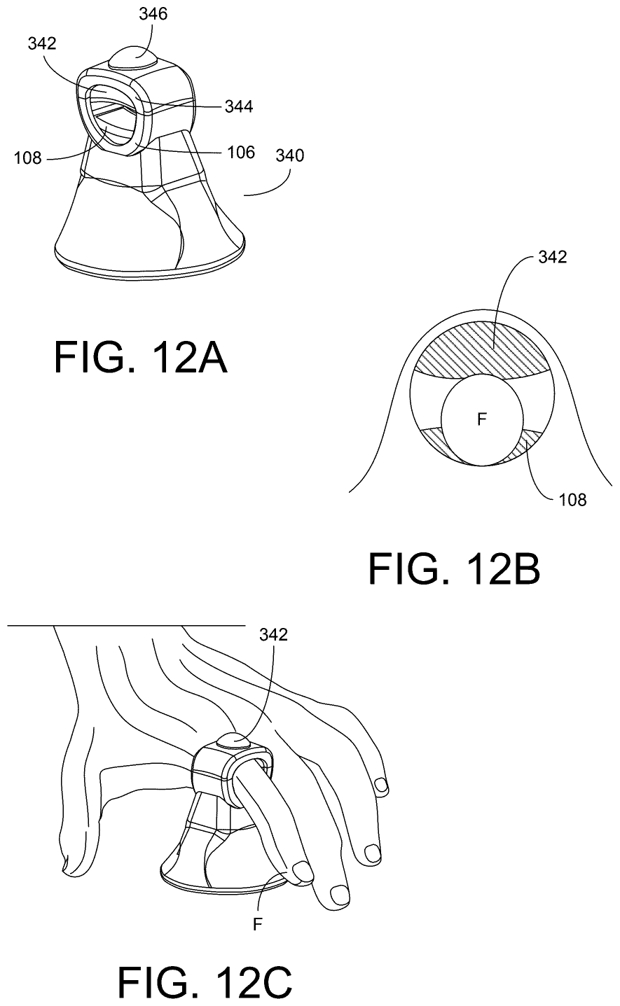

The VSMD 340 of FIGS. 12A-12 C employs an SOI implementation that comprises a second inflatable bladder 342 disposed at the top of a cuff formed by cradle 106 (on which the inflatable measurement bladder 108 is disposed) and an upper portion 344. When inflated, the second bladder 342 applies the downward pressure/force. A bolus pump 346 disposed on the upper portion 344 (or elsewhere) may be employed to inflate the second bladder 342 prior to commencing a blood pressure measurement cycle. In one implementation, after the finger F has been inserted into the cuff, the bolus pump is used to inflate the second bladder to about 10 mm Hg to about 50 mm Hg. This is a range that has been found to sufficiently retrain movement of the finger pad relative to the inflatable bladder. Thereafter, the inflatable bladder is inflated/deflated as herein described. A manual relief valve (not shown) may be employed to deflate the second bladder following the conclusion of a blood pressure measurement cycle.

The SOI implementation for the VSMD 360 of FIG. 13 is similar in concept that of FIGS. 12A-12C in that a second inflatable bladder is employed to apply the downward force/pressure. A syringe 362 is pneumatically coupled to the second bladder via an air hose 364. A manual valve 366 may be employed to maintain pressure in the second bladder during a blood pressure measurement cycle and to deflate the second bladder following the conclusion thereof. As shown, if desired, shims a-n may also be employed as described, albeit in conjunction with inflation of the second bladder.

In yet another implementation, not illustrated, the downward force or pressure may be applied by pressing down on the portion of the top of finger F from which blood pressure measurement is to be taken by way of a finger on the user's other hand, such that lateral movement of the finger pad on finger F is limited to a range of about 0.4 mm-4 mm relative to the surface of the inflatable bladder that contacts the finger pad.

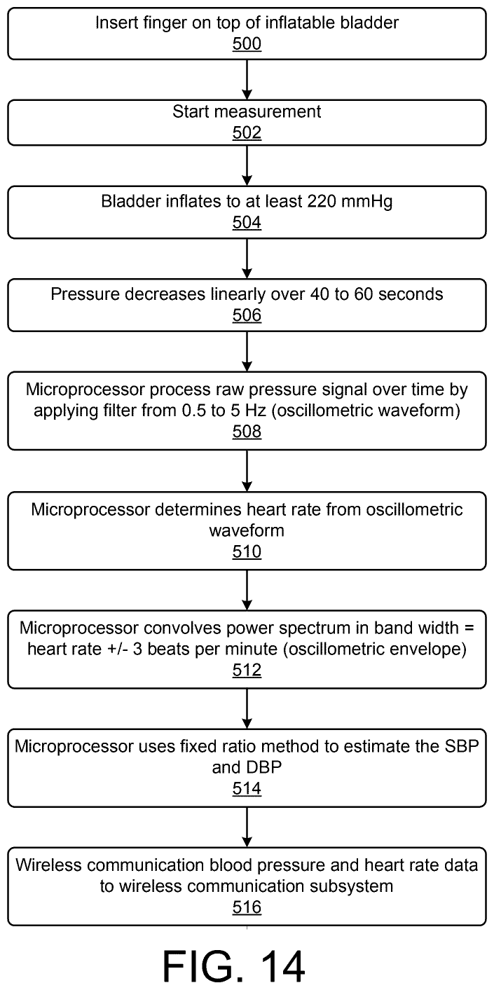

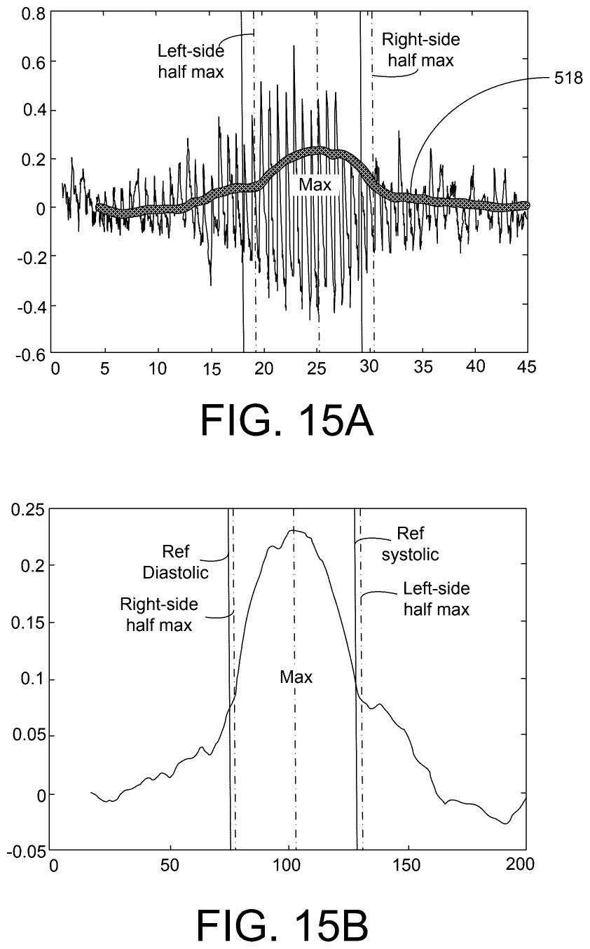

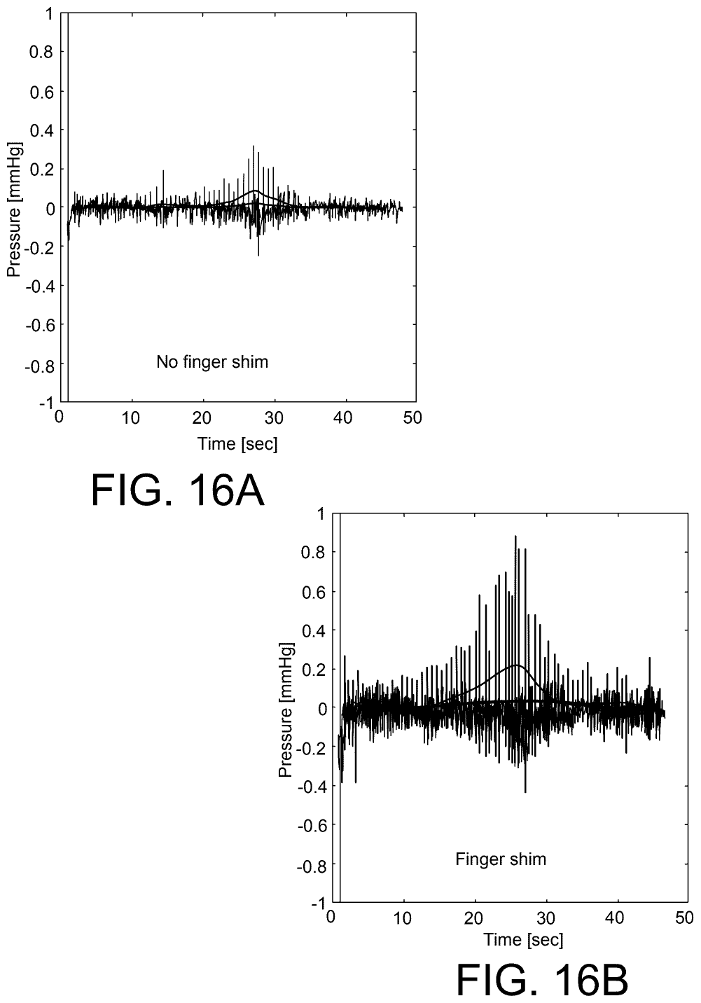

Referring to FIG. 14, there is shown a method of determining blood pressure, i.e., systolic blood pressure (SBP) and diastolic blood pressure (DBP) and pulse rate via the pressure sensor data. After a finger has been inserted onto the bladder 108 and the appropriate amount of downward pressure/force has been applied by the SOI, measurements may begin (502). Once a measurement cycle has begun, the pump inflates the bladder 108 to a target pressure, which is generally at least 220 mmHg (504). Once the bladder has reached its target pressure, the relief valve is controllably opened to allow the bladder pressure to decrease in a linear fashion over a period of 40-60 seconds (506). All of the foregoing steps are carried out under control of microprocessor 146 executing appropriate program code. As the bladder is deflated, the microprocessor processes raw pressure signal data over a period of time by applying a 0.5 Hz to 5 Hz filter thereto, to yield the oscillometric waveform (FIG. 15A). The oscillometric waveform data is analyzed by the microprocessor to provide an indication of heart rate (510). Heart rate data is then processed by the microprocessor to create a power spectrum 518 (FIG. 16A), and the microprocessor then convolves the power spectrum as noted at 512. The microprocessor then employs the fixed ratio method to provide an indication of SBP and DBP (514). Blood pressure and heart rate data is then communicated to a subsystem for storage and later use, either within the VSMS or remote from the VSMD, e.g., the RPMS (516). The SOI is thereafter released, removed, or deflated, so to allow removal of the finger.

FIG. 15A is one example of an oscillometric waveform vs time (seconds). FIG. 15B is one example of an oscillometric waveform envelope vs. pressure (mmHg). The fixed-ratio method is employed to estimate systolic and diastolic blood pressure values from this data.

FIGS. 16A and 16 B illustrate pressure sensor amplitude data, as measured in the inflatable measurement bladder, during inflation and deflation, without the use of an SOI (FIG. 16A) and with the use of an SOI in conjunction with VSMD embodiments such as disclosed herein (FIG. 16B). It will be seen that use of the embodiments such as described herein significantly and substantially improve blood pressure measurement results.

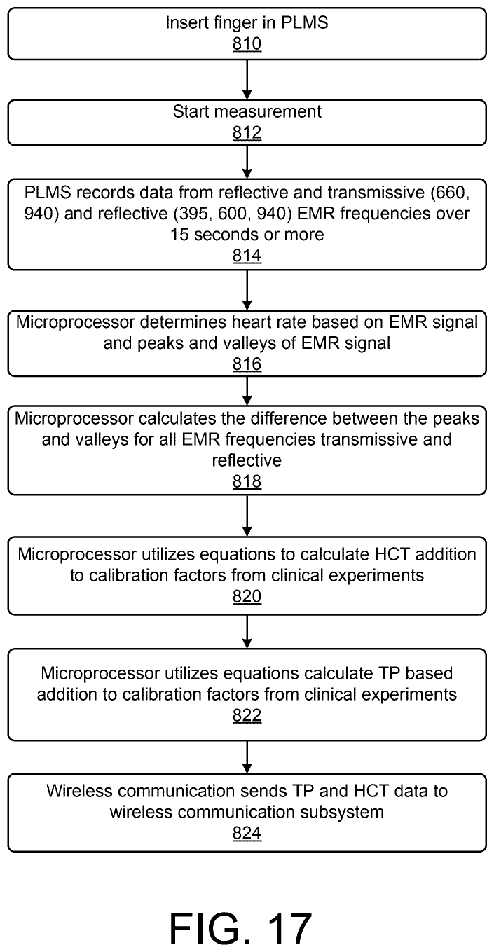

FIG. 17 illustrates a process flow for measuring Hc and TP employing the systems and methods described herein. After a finger has been inserted into the PLMS (810), measurements may begin (812) either automatically by way of finger detection using a proximity sensor, or via user initiation. Thereafter, the PLMS records transmissive and reflective data from the detectors for a period of 15 seconds or more (814). The microprocessor determines heart rate (816) and calculates differences between peaks and valleys in the detected EMR data (818). The microprocessor then calculates Hc (820) and TP (822) and described above. The TP and Hc are then transmitted to a communications subsystem (824). Further details are set forth in the 014 patent.

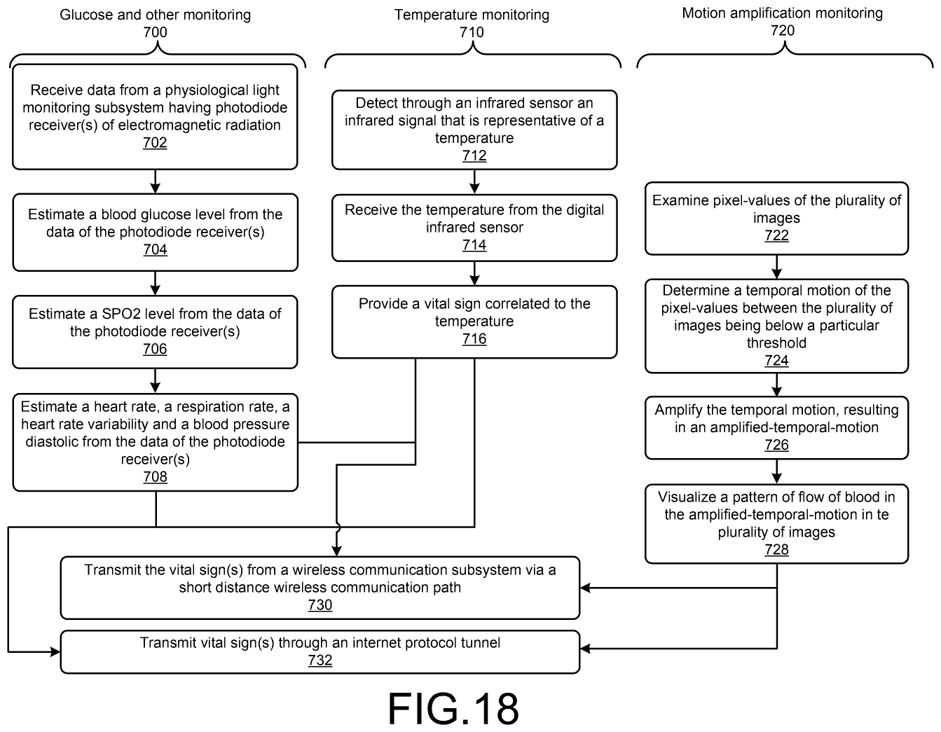

Referring to FIG. 18, method 700, may be used for measuring blood glucose and other physiological characteristics/vital signs include receiving data from a SpO2/glucose subsystem having photodiode receivers of ER (702). One example of the SpO2/glucose subsystem is PLMS 204. Blood glucose levels and SpO2 can be derived via the data received from the detector 222 (704, 706). Heart rate, respiration rate, heart rate variability and DBP may also be derived (708). Method 710 may be used for detecting an infrared signal that is representative of a body surface temperature (712), receiving the body surface temperature from the infrared sensor (714), and providing a data such as body core temperature correlated to the body surface temperature (716). Method 720 may be used to examine pixel values of a plurality of images (722) of the finger, determining a temporal motion of the pixel values between the plurality of images being below a particular threshold (724), amplifying the temporal motion, resulting in an amplified temporal motion (726), and visualizing a pattern of flow of blood in the amplified temporal motion in the plurality of images (728).

The measured vital signs may be transmitted from a communication subsystem, e.g., via a short distance wireless communication path (730), and/or securely to a RPMS (732).

Measurement of Hc and TP may also employ the PLMS and an alternate version of method 700. In this case, data from a reflective 395 nm emitter is used to calculate TP via a ratio with data from a reflective 940 nm emitter on side of the finger opposite the fingernail. The hematocrit is calculated via the transmissive 660 nm and 940 nm signals via the equation below where f_{w} and fₚₚ are parameters established through clinical trial calibration with the patients.

$$\frac{1}{H} \tilde{=} \frac{0.34}{1 - {f}_{w} - {f}_{pp}} ( 1 + R \frac{μ {}_{a}^{Hb} ( {λ}_{1} ) + {Δμ}_{s} ( {λ}_{1} )}{μ {}_{a}^{w} ( {λ}_{2} ) + {Δμ}_{s} ( {λ}_{2} )} ) .$$

where:
H is the hematocrit value (Hc);
fw is the tissue water fraction;
fpp is the plasma protein fraction;
R is the ratio of magnitudes of the blood pulse spectrum;
μaHb(λ1) is the sum of the absorption coefficient of the two forms of hemoglobin at
a first wavelength;
μaw(λ2) is the absorption coefficient of water at a second wavelength;
Δμs(λ1) is the difference between the scattering coefficients of the blood and surrounding tissue at a first wavelength;
Δμs(λ2) is the difference between the scattering coefficients of the blood and surrounding tissue at a second wavelength; and
0.34 is the fraction of the red cell volume occupied by hemoglobin, which is assumed to be constant.

The VSMD's described herein may be equipped to communicate with a smartphone, via wired (USB port connection) or wireless (Bluetooth, WiFi, etc.) having an appropriate app installed thereon. Via the app, data can be uploaded from the VSMD to the smartphone for use by the app, and/or can be further uploaded to the RPMS. Via the app, firmware updates, feature upgrades, etc., to the VSMD may also be carried out.

The apparatuses, systems and methods described herein may be embodied in other specific forms without departing from the spirit or essential attributes thereof. Accordingly, reference should be made to the appended claims, rather than the foregoing specification, for indicating the scope of the innovations described herein.

1. An apparatus for facilitating measurement of blood pressure via a pad on the underside of a human finger, comprising:
a. a flexible inflatable blood pressure measurement bladder (“measurement bladder”) having a surface adapted to contact the underside of the finger, defining a finger pad contact surface and being adapted to apply pressure to the finger pad when inflated;
b. adjustable force application structure (“force structure”), separate from the measurement bladder, adapted to adjustably apply a force to a portion of an upper side of the finger opposite the finger pad and press the finger pad against the finger pad contact surface;
c. a measurement bladder pump in pneumatic communication with the measurement bladder for inflating the bladder;
d. a relief valve in pneumatic communication with the measurement bladder for deflating the measurement bladder;
e. a sensor for providing pressure data indicative of air pressure in the measurement bladder; and,
f. a control system for controlling the operation of the measurement bladder pump and the relief valve by: (i) causing the measurement bladder pump to inflate the measurement bladder sufficiently to urge the finger pad contact surface against the finger pad and to urge the upper side of the finger against the force structure; (ii) causing the measurement bladder pump to stop inflation of the measurement bladder when the pressure data indicates that a predetermined inflation pressure, that has been determined to substantially stop the flow of blood in arteries of the finger pad (inflation pressure), has been reached; and thereafter (iii) causing the relief valve to controllably deflate the measurement bladder.

2. The apparatus according to claim 1 where the measurement bladder has a surface adapted to contact no more than the underside of the finger.

3. The apparatus according to claim 1 wherein the control system comprises program instructions for: detecting resumption of blood flow in the arteries by monitoring the pressure data during deflation and detecting pressure oscillations therein; detecting when normal flow has resumed via the pressure oscillations and the pressure at which normal blood flow resumes (deflation pressure); and calculating an indication of blood pressure based on the inflation and deflation pressures.

4. The apparatus of claim 1 further comprising a substantially rigid arcuate cradle, the measurement bladder having an underside, opposite the finger pad contact surface, secured to the cradle such that the measurement bladder substantially conforms to the shape of at least a portion of the arc of the cradle.

5. The apparatus of claim 1 further comprising a slotted structure disposed on one side of the cradle and wherein the force structure comprises a hook and latch fastening strap secured to an opposing side of the cradle, the fastening strap adapted to be looped over a finger that has been placed in the cradle, through the slotted structure, and back over and fastened to itself so as to apply the force to the finger.

6. The apparatus of claim 1 wherein the force structure further comprises an arcuate cap flexibly connected to the cradle and adapted to form a closure over the cradle so as to define a cavity adapted to allow receipt of the finger between an inner surface of the cap and the finger pad contact surface, the fastening strap extending over an outermost surface of the cap such that the strap applies the force to the finger via the cap.

7. The apparatus of claim 6 wherein the cap is hinged to one side of the cradle such that the cap may be rotated to expose the finger pad contact surface and allow receipt of the finger.

8. The apparatus of claim 6 wherein the cap is flexibly connected to opposing sides of the cradle so as to allow up and down movement of the cap relative to the measurement bladder.

9. The apparatus according to claim 1 wherein the force structure comprises one or more shims and wherein the arc of the cradle forms a complete circle so as to define a cavity, the measurement bladder being disposed only in a bottom portion of the cavity, an upper portion of the cavity being adapted to receive one or more of the shims.

10. The apparatus according to claim 1 wherein the force structure comprises a bladder separate and independent of the measurement bladder (“force application bladder”) and wherein arc of the cradle forms a complete circle so as to define a cavity, the measurement bladder being disposed only in a bottom portion of the cavity, the force application bladder being disposed in an upper portion of the cavity, such that inflation of the force application bladder presses the finger against the finger pad contact surface.

11. The apparatus of claim 10 further comprising a force application bladder pump, separate and independent of the measurement bladder pump, for inflating the force application bladder.

12. The apparatus of claim 11 wherein the force application bladder pump comprises a syringe pneumatically coupled to the force application bladder.

13. The apparatus of claim 11 wherein the force application pump comprises a bolus pump integral with the cradle.

14. The apparatus of claim 1 wherein the force structure comprises an arcuate cap flexibly connected to the cradle and adapted to form a closure over the cradle so as to define a cavity adapted to allow receipt of the finger between an inner surface of the cap and the finger pad contact surface, a strap extending from one side of the cavity, over or through the cap to an opposing side of the cavity, and an adjustment mechanism adapted control a tension applied to the cap via the strap and thereby apply the force pressure.

15. The apparatus of claim 3 wherein there are no electromagnetic radiation (EMR) sensors employed to provide the indication of blood pressure and no EMR data is employed to calculate the indication of blood pressure.

16. The apparatus of claim 3 wherein the blood pressure indication is calculated based solely on the pressure data measured from the pressure sensor.

17. The apparatus of claim 3 further comprising a light emitting and light detecting system for detecting amounts of light transmitted through and reflected from a forefinger portion of the finger, and the program instructions include instructions for calculating indications of vital signs based on the amounts of detected transmitted and reflected light, the other indications comprising one or more of blood glucose level, respiration rate, saturation of peripheral oxygen, blood flow, total hemoglobin, pleth variability index, methemoglobin, acoustic respiration rate, carboxyhemoglobin, oxygen reserve index, oxygen content, nitric oxide, microvascular blood flow, EEG hematocrit, and total protein.

18. The apparatus of claim 1 wherein the force structure is adapted to apply sufficient pressure to the upper side of the finger so as to limit lateral movement of the finger pad to a range of about 0.4 mm to about 4 mm relative to the finger pad contact surface.

19. An apparatus for facilitating measurement of blood pressure (BP) of a person via a pad of the underside of a finger comprising:
a) a base having a top and a bottom;
b) a substantially inflexible arcuate cradle affixed to the top of the base so as to facilitate receipt of the finger;
c) a flexible inflatable blood pressure measurement bladder (“measurement bladder”) having an underside secured to the cradle such that the measurement bladder substantially conforms to the shape of at least a portion of the arc of the cradle, an upper side of the bladder defining a finger pad contact surface and being adapted to apply pressure to the finger pad when inflated;
d) adjustable force application structure (“force structure”), separate from the measurement bladder, adapted to adjustably apply a force to an upper side of the finger and press the finger pad against the finger pad contact surface sufficient to limit lateral movement of the finger pad relative to the finger pad contact surface to a range of about 0.4 mm to about 4 mm;
e) a measurement bladder pump and a measurement bladder relief valve for inflating and deflating the measurement bladder, respectively, a pressure sensor for providing data indicative of air pressure and air pressure oscillations in the measurement bladder, each of the measurement bladder pump, measurement bladder relief valve and pressure sensor disposed in the base;
f) there being no light emitter or detector systems employed to provide an indication of BP;
g) a control system disposed in the base and coupled to the measurement bladder pump, the measurement bladder relief valve and the pressure sensor, the control system including a microprocessor and a memory having program instructions stored therein for execution by the microprocessor for controlling the operation of the measurement bladder pump and the measurement bladder relief valve so as to:
i. inflate the measurement bladder such that the finger pad contact surface is urged against the finger pad and the upper side of the finger is urged against the force structure; and,
ii. stop measurement bladder inflation when a predetermined inflation pressure, that has been determined to substantially stop the flow of blood in arteries of the finger pad (inflation pressure), has been reached and thereafter controllably deflating the measurement bladder.

20. The apparatus of claim 19 wherein the program instructions cause the control system to detect resumption of blood flow in the arteries by monitoring the pressure data and detecting pressure oscillations therein; and to detect when normal flow has resumed via the pressure oscillations, and the pressure at which normal blood flow resumes (deflation pressure).

21. The apparatus according to claim 20 wherein the program instructions further calculate an indication of blood pressure based on the inflation and deflation pressures by generating a heart rate power spectrum from the waveform, then next convolving the power spectrum, and then next applying a fixed ratio method to provide an indication of systolic and diastolic BP; and wherein none of the steps for measuring blood pressure employ data from a light emitter or a light detector.

22. The apparatus according to claim 19 wherein the force structure comprises a slotted structure disposed on one side of the cradle and a hook and latch fastening strap secured to an opposing side of the cradle, the fastening strap adapted to be looped over a finger that has been placed in the cradle, through the slotted structure, and back over and fastened to itself so as to apply the force to the finger.

23. The apparatus according to claim 19 wherein the force structure comprises an arcuate cap flexibly connected to the cradle and adapted to form a closure over the cradle so as to define a cavity adapted to allow receipt of the finger between an inner surface of the cap and the finger pad contact surface, the fastening strap extending over an outermost surface of the cap such that the strap applies the force to the finger via the cap.

24. The apparatus according to claim 19 wherein the force structure comprises one or more accurate shims, wherein the arc of the cradle forms a complete circle so as to define a cavity, the measurement bladder being disposed only in a bottom portion of the cavity, an upper portion of the cavity being adapted to receive one or more of the shims.

25. The apparatus according to claim 19 wherein the force structure comprises a bladder separate and independent of the measurement bladder (“force application bladder”) and wherein the arc of the cradle forms a complete circle so as to define a cavity, the measurement bladder being disposed only in a bottom portion of the cavity, the force application bladder being disposed in an upper portion of the cavity, such that inflation of the force application bladder presses the finger against the finger pad contact surface.

26. The apparatus according to claim 19 wherein the force structure comprises an arcuate cap flexibly connected to the cradle and adapted to form a closure over the cradle so as to define a cavity adapted to allow receipt of the finger between an inner surface of the cap and the finger pad contact surface, a strap extending from one side of the cavity, over or through the cap to an opposing side of the cavity, and an adjustment mechanism adapted control a tension applied to the cap via the strap and thereby apply the force pressure.

27. The apparatus according to claim 19 further comprising a light emitting and light detecting system (PLMS) for detecting amounts of light transmitted through and reflected from a forefinger portion of the finger, and program instructions for calculating indications of vital signs based on the amounts of detected transmitted and reflected light, the other indications comprising one or more of blood glucose level, respiration rate, saturation of peripheral oxygen, blood flow, total hemoglobin, pleth variability index, methemoglobin, acoustic respiration rate, carboxyhemoglobin, oxygen reserve index, oxygen content, nitric oxide, microvascular blood flow, EEG hematocrit, and total protein, the PLMS comprising a housing for receiving the finger disposed on the top of the base in substantial axial alignment with the cradle such that BP measurements are taken at a proximal end of the finger and the PLMS measurements are taken at a distal end of the finger.

28. A method of using an apparatus that comprises:
a. a substantially inflexible arcuate cradle;
b. a flexible inflatable blood pressure measurement bladder (“measurement bladder”) having an underside secured to the cradle such that the measurement bladder substantially conforms to the shape of at least a portion of the arc of the cradle, an upper side of the bladder defining a finger pad contact surface and being adapted to apply pressure to a human finger pad when inflated;
c. a measurement bladder pump in pneumatic communication with the measurement bladder for inflating the bladder;
d. a relief valve in pneumatic communication with the measurement bladder for deflating the measurement bladder;
e. a pressure sensor for providing data indicative of air pressure in the measurement bladder; and,
f. a control system for receiving the data from the pressure sensor and for controlling the operation of the measurement bladder pump and relief valve by:
i. inflating the measurement bladder such that the finger pad contact surface is urged against the finger pad; and,
ii. stopping measurement bladder inflation when a predetermined inflation pressure, that has been determined to substantially stop the flow of blood in arteries of the finger pad (inflation pressure), has been reached and thereafter controllably deflating the measurement bladder;
the method comprising:
g. inserting a finger into the cradle such that the finger pad contacts the finger pad contact surface;
h. applying a force to a portion of the finger opposite the finger pad so as to urge the finger pad against the finger pad contact surface in an amount no more than is required to prevent the finger from moving no more than about 0.4 mm and less than about 4 mm laterally with respect to finger pad contact surface; and,
i. causing the control system to initiate an operation that carries out steps (f)(i)-f(ii).
